(19) United States
(12) Patent Application Publication
(10) Pub. No.: US 2012/0258147 A1
(43) Pub. Date: Oct. 11, 2012

(54) METHOD FOR INHIBITING ANGIOGENESIS

(75) Inventors: Juergen Mollenhauer, Reutlingen (DE); Burkhard Schlosshauer, Tuebingen (DE); Beate Scholz, Dassenheim (DE)

(73) Assignee: NMI Naturwissenschaftliches und Medizinisches Institut an der Universitaet Tuebingen, Reutlingen (DE)

(21) Appl. No.: 13/449,798

(22) Filed: Apr. 18, 2012

Related U.S. Application Data
(63) Continuation of application No. PCT/EP2010/066158, filed on Oct. 26, 2010.

(30) Foreign Application Priority Data
Oct. 26, 2009 (DE) 102009051575.5

(51) Int. Cl. A61K 38/38; A61K 9/00; A61P 19/04; A61P 27/02; A61P 35/00; A61P 19/02; A61P 37/00; A61P 9/10; A61P 3/04; A61P 17/06; A61P 9/12; A61P 11/06; A61K 35/12; A61P 31/00

(52) U.S. Cl. 424/400; 514/13.3; 424/93.7

(57) ABSTRACT

The present invention relates to a method for inhibiting and/or preventing angiogenesis, the method comprising the step of administering a biocompatible composition, which is polymerizable to a hydrogel-forming material and which is based on a hydrophilic polymer, for inhibiting and/or preventing angiogenesis or endothelial cell proliferation, wherein the hydrophilic polymer is crosslinkable serum albumin or crosslinkable serum protein, in a subject in need of being treated. The invention furthermore relates to a method for inhibiting and/or preventing angiogenesis or endothelial cell proliferation in a subject in need thereof, comprising the step of administering a polymerized hydrogel-material, which has been obtained by polymerizing a serum-albumin- or serum-protein-based composition.

### CROSS REFERENCES TO RELATED APPLICATIONS

This application is a continuation of international patent application PCT/EP2010/066158, filed on Oct. 26, 2010 designating the U.S., which international patent application has been published in German language and claims priority from German patent application DE 10 2009 051 575.5, filed on Oct. 26, 2009. The entire contents of these priority applications are incorporated herein by reference.

### BACKGROUND OF THE INVENTION

The present invention relates to a method for inhibiting and/or preventing angiogenesis or endothelial cell proliferation.

What is referred to as angiogenesis is—generally speaking—the development of novel vascular structures which are lined by endothelial cells and also include smooth muscle cells and pericytes. Angiogenesis plays an important role not only in physiological processes, for example in embryonal development and wound healing, but also in pathological processes, for example in polyarthritis and tumor growth.

Research and literature has used and still uses three different expressions for the regeneration of vessels in some cases—vasculogenesis, angiogenesis and arteriogenesis—with the expression angiogenesis currently being accepted as the umbrella term for all forms of revascularization since a delimitation of the three abovementioned forms is difficult in some cases and the underlying principle is the same.

Angiogenesis is a complex process in which the endothelial cells, pericytes and smooth muscle cells required for producing the vessel walls are activated by various angiogenetic growth factors, for example by the fibroblast growth factor (FGF) and/or the vascular endothelial growth factor (VEGF). New capillaries are produced by the proliferation and migration of endothelial cells which already exist in the tissue in question.

Angiogenesis is of considerable biological and medicinal importance; one distinguishes between two therapeutic uses of angiogenesis, the pro-angiogenetic treatment and the anti-angiogenetic, or non-angiogenetic, treatment.

In the first case, it is intended to stimulate vascular regeneration, in particular by employing and administering growth factors, such as, for example, for treating arteriosclerosis, in particular coronary heart disease and peripheral arterial occlusive disease.

Anti-angiogenic or non-angiogenic treatment is employed in particular where vascular regeneration is prevented at all costs and is undesired, such as, for example, in the treatment of tumors, since solid tumors depend on a simultaneously growing capillary network which supplies the tumor with oxygen and nutrients. Accordingly, anti-angiogenetic therapeutic approaches attempt to reduce/block the vascular supply and thus the blood flow of a tumor. Thus, for example, VEGF-neutralizing monoclonal antibodies have been employed in the prior art for an anti-angiogenetic treatment of tumors.

In other diseases too, such as Crohn's disease, psoriasis and rheumatoid arthritis, unhindered angiogenesis plays an important role since the newly generated vessels provide a constant supply of inflammatory cell populations to the affected places in the body. An overview of diseases and afflictions which are directly related to angiogenesis can be found, for example, in table 1 of the publication by Polverini, “Angiogenesis in health and Disease: Insights into Basic Mechanisms and Therapeutic Opportunities”, Journal of Dental Education, (2002) vol. 66, 962-975.

In implantable medical devices/implants too, that is to say, for example, implants by which damaged tissue is to be replaced, or in stents/stent grafts, which are introduced into specific organs so as to support their walls, the prerequisite for permanent successful use is frequently that these devices do not promote vascular regeneration at the place where they have been implanted, but that they are inserted as neutrally and as inertly as possible into the tissue surrounding them, where they are also resorbed under certain circumstances. In these cases there is the grave risk in medical implants that endothelial cells adhere to the latter, thereby starting up the mechanisms of vascular regeneration. This may result in undesired side effects such as, for example, swelling and consolidation of the tissue into which the device has been implanted, as far as the growth of tumors.

To prevent this, the devices to be implanted are, in the prior art, frequently coated with anti-angiogenic (and also anti-inflammatory) active substances such as, for example, antibodies (for example anti-VEGF antibodies), retinoic acid and its derivatives, suramine, metal proteinase-1 and metal proteinase-2 inhibitors, epothilone, colchicine, vinblastine, paclitaxel and the like, which are intended to inhibit the adhesion of endothelial cells on the devices, and the vascular regeneration which can be triggered thereby.

However, the disadvantage of devices/implants coated thus is firstly that the production is complicated due to the additional coating step and secondly that the anti-angiogenic or non-angiogenic activity of the coating depends on the quality/quantity of the application of the coating and of the active substance, and on the durability of the coating. Furthermore, it has emerged in the past that even coated implants were not capable of fully preventing the adhesion of endothelial cells. In addition, the coatings frequently cause side effects in the patient to be treated, which influence not only the success of the intervention in question but which may also overall be damaging to the patient's health.

Therefore, there continues to be a large demand for providing medical implants and methods for inhibiting angiogenesis with which the disadvantages of the prior art can be overcome and which, while being efficient to use and inexpensive to prepare, have outstanding biocompatibility at the same time.

### SUMMARY OF THE INVENTION

Against this background, it is an object of the present invention to provide novel methods for inhibiting and/or preventing angiogenesis or endothelial cell proliferation and provide means for a medical implant which do not trigger and/or cause vascular regeneration and/or which inhibit the adhesion of endothelial cells.

The object is achieved in accordance with the invention by a method for inhibiting and/or preventing angiogenesis or endothelial cell proliferation in a subject in need thereof, in particular in an amount effective to inhibit and/or prevent angiogenesis or endothelial cell proliferation, wherein the method comprises the step of administering a biocompatible composition which is based on a hydrophilic polymer and which is polymerizable to a hydrogel(-forming)material, and wherein the hydrophilic polymer is crosslinkable serum albumin or crosslinkable serum protein.

The invention furthermore also relates to a method for coating and for modifying the surfaces of implants, which consist of materials other than the material which is polymerized starting from the abovementioned composition, wherein in the coating method the abovementioned biocompatible composition is applied as a coating.

The object is furthermore achieved by a method for inhibiting and/or preventing angiogenesis or endothelial cell proliferation in a subject in need thereof comprising the step of administering a polymerized hydrogel(-forming) material which has been obtained by polymerizing a serum-albumin- or serum-protein-based composition, in particular in an amount effective to inhibit and/or prevent angiogenesis or endothelial cell proliferation.

The methods according to the invention comprising the step of administering the polymerizable composition or the polymerized hydrogel-material provide a novel therapeutic means and/or a medical support material which allow, for example, replacement of tissues by means of an implant while simultaneously inhibiting the adhesion and proliferation of endothelial cells thereon. This advantageously avoids vascular regeneration and swelling and consolidation of the tissue into which the composition for replacing a diseased or defective tissue is introduced, while simultaneously replacing the defective or diseased tissue by resorption of the material.

The method according to the invention thus provides a support material for an implant, by means of which support material angiogenesis can be inhibited in a deliberate fashion, and for example the growth of other cells which are not involved in angiogenesis can be promoted in a deliberate fashion by previously having been introduced into the composition/the material.

A further advantage of the novel method is that the composition may also be polymerized only when in situ, in other words the composition can be injected at the site where it is desired to replace and/or support a tissue, and only then polymerizes fully at this site. This means that the claimed therapeutic treatment only requires minimal medical intervention. On the other hand, the composition may also be polymerized fully before being introduced into a patient's body and then be implanted by means of a surgical intervention.

The inventors have demonstrated that a serum-albumin- and/or serum-protein-based composition and/or the material obtained by polymerization thereof are outstandingly suitable as support material for inhibiting the adhesion of endothelial cells and therefore for inhibiting/preventing angiogenesis. Thus, the serum-albumin-/serum-protein-based composition/material can, e.g., be employed as a medical implant for inhibiting angiogenesis in particular where vascular regeneration is disadvantageous and/or must be prevented at all costs, for example in the case of a tissue substitute of cartilage, intervertebral disks, cornea. Surprisingly, it has been shown in the experiments on which the invention is based that only the serum-albumin-/serum-protein-based material inhibits adhesion of endothelial cells in comparison with other known supports or matrices used in the prior art. Here, the material itself is not toxic to the endothelial cells—and therefore also not toxic to the patient who is to receive the material, e.g. as a medical implant—, which in turn demonstrates that the biocompatibility of the material for the patient is particularly high.

In addition, serum albumins are capable of binding a large number of different substances such as, for example, metal ions (metals), fatty acids and amino acids, various proteins and pharmaceuticals, which is why they are extremely biocompatible and therefore cause virtually no reactions in the body.

Therefore, the method according to the invention may also be implemented in combination with the administration of other biologically and/or therapeutically active substances which, via the composition and/or the hydrogel-material are intended to have a biological and/or therapeutic effect at the target site of the patient. In this context, the method according to the invention can be implemented in such a way that the material is polymerized only when in situ or else is already polymerized before the implanting procedure and is implanted in the hydrogel state. In this context, naturally, a somewhat more solid consistency of the hydrogel is preferred when the fully polymerized hydrogel is implanted, and this somewhat more solid consistency makes possible, or facilitates, practical handling of the hydrogel. The degree of the solidity, or the fluid property of the hydrogel and/or of the material, can in this context be adjusted via the degree of crosslinking of the hydrogel and/or of the material, the hydrogel and/or the material being the more solid the more it is crosslinked. Thus, the fluid properties of a gel are between that of a liquid and that of a solid body.

Within the present context, and as generally in the state of the art, with a “hydrogel” or hydrogel-forming” material is meant a water-unsoluble hydrophilic polymer the molecules of which are chemically—e.g. via covalent or ionic bonds—or physically—e.g. by interlacing the polymer chains—connected to form a three-dimensional network.

Although albumin is known as a biocompatible substance and also described as a gel and/or support material as such, for example, in DE 10 2008 008 071.3, its use for inhibiting the adhesion and proliferation of endothelial cells and for inhibiting angiogenesis was not known.

The composition to be employed or administered in the method according to the invention and/or the polymerized hydrogel-forming material based thereon can, in this context, include serum albumin/serum proteins which are obtained from any mammal and/or accordingly can be employed/administered for any mammal, wherein human, bovine, ovine, rabbit serum albumin are preferred and wherein the method according to the invention is preferably implemented in humans using a material based on human serum albumin.

A further advantage of the method according to the invention is that the precursor of the hydrogel-forming material may be handled at room temperature. Accordingly, the material can be stored separately from eventually to be co-administered additives or cells to be introduced where necessary and combined shortly before the method according to the invention with the additives, if desired, or optionally cells intended to support for example tissue regeneration. In this context, the polymerization time is adjustable, it being possible to provide times of between a few seconds and 2 minutes for polymerization. Therefore, the additives and/or cells are immediately anchored in the material so that undesired diffusion from the material is avoided. In this context, and as has already been mentioned above, the hydrogel-material can be administered so that it polymerizes in situ, or else, the already polymerized material can be administered and/or introduced into a patient's body.

In the present application, the expressions “composition” and “material” are used for the method according to the invention, where “composition” is used predominantly, but not exclusively, for the as yet unpolymerized material, and “material” or “gel”/“hydrogel” for the polymerized composition. Even so, it is understood that these expressions cannot be separated fully from each other since the composition and the material actually mean the same object. In this context, “gel”/“hydrogel” is understood as meaning the semi-solid state of the composition which is present in the form of a three-dimensional polymerized network.

A further advantage of the method according to the invention is that the basic material for the hydrophilic polymer is variable so that, on the one hand, commercially available albumin, for example human albumin, purified or recombinantly produced, may be employed, and, on the other hand, also allogenous or autologous serum.

As already mentioned, the method according to the invention is implemented in such a way that the serum-albumin- and/or serum-protein-based composition is injected into the site to be treated, where it polymerizes into the hydrogel-material, or else the polymerized hydrogel-material is implanted directly. After the introduction into the patient, the crosslinked albumin dissolves within a specific period of time, during which time for example cells, if present in the material, have developed in situ a pericellular matrix and thus become embedded into the environment. At the same time, this prevents endothelial cells from adhering and proliferating and thus triggering vascular regeneration starting from the material.

In this context, one embodiment of the method according to the invention provides that the albumin concentration in the polymerized hydrogel-material is from approximately 5 to approximately 20, in particular approximately 10 mg/ml of material.

Examples of methods for preparing the composition for the use according to the invention can be found in DE 10 2008 008 071.3, which has already been mentioned hereinabove and whose content is herewith expressly referred to.

According to the invention, a preferred embodiment provides that, for example, live mammalian cells, in particular live human cells, and a pharmacological agent, a biologically active agent, or one or more or mixtures of these are used together with the composition/material.

In this context, mammalian cells are understood as meaning any cell which is derived or originates from a mammal, this expression encompassing in particular human and animal cells. Such cells can be selected for example among musculoskeletal cells, in particular chondrocytes, osteocytes, fibrochondrocytes, and metabolism-regulating glandular cells, islet cells, melatonin-producing cells, precursor cells and stem cells, in particular mesenchymal stem cells, in other words cells which are suitable and desired for the respective method comprising the step of administering the composition and/or for the respective injection site. These cells are viable in the composition and/or the polymerized hydrogel-forming material and regenerate tissue while simultaneously resorbing the material.

The method according to the invention is also suitable for preventing vascular regeneration in therapies whose aim is the hormone production in situ, such as, for example, insulin, thyroxin or melatonin. When cells which produce these hormones or other hormones are introduced into the site to be treated in the body of a patient, via the composition or the material, they produce the hormones in question and secrete them into the environment, with the simultaneous prevention of vascular regeneration.

Naturally, the method according to the invention may also be implemented/administered as a combined effect together with biological or pharmaceutically active substances. “Biologically active or effective substance” and “pharmaceutically active or effective substance” is understood as meaning, in this context, any natural or synthetic substance which either can exert a biological or pharmaceutical influence on cells or tissue or can effect the reactions on or in cells. In this context, this influence may be limited to specific cells and specific conditions without the substance losing its biologically or pharmaceutically active meaning. The chemical nature of the substances which can be used here is, in this context, not limited to a specific class (of compounds); rather, it may include any natural and synthetic substance which in its natural form and/or in modified form has any effect on biological cells.

Thus, it is especially preferred to employ for example antibiotics, anti-inflammatories, metabolism hormones, chondroprotectants, agents for gene therapy, growth hormones or differentiation and/or modulation factors, immunosuppressants, immunostimulants, generally peptides, proteins, nucleic acids, organic active substances, hyaluronic acid, apoptosis-inducing active substances, receptor agonists and receptor antagonists, or mixtures of these as biologically or pharmaceutically active or effective substances. Furthermore, it is possible to employ extracellular matrix proteins, cell surface proteins, and generally polysaccharides, lipids, antibodies, growth factors, sugars, lectins, carbohydrates, cytokins, DNA, RNA, siRNA, aptamers and binding- or activity-relevant fragments thereof, and what are referred to as disease-modifying osteoarthritis agents, or mixtures of these. In this context, all substances can be synthetically produced or naturally occurring or originate from recombinant sources. In this context, “disease-modifying osteoarthritis agents” (DMOAs) are understood as meaning a series of substances which are currently employed as medicament, in particular for arthrosis—but in the meantime also in other autoimmune diseases—for alleviating disease and inflammation, and whose precise mechanism of action has so far not been elucidated fully. Most of these substances comprise mixtures of glucosamine and chondroitin sulfate.

In particular, it is preferred in one embodiment of the method according to the invention if the biologically active substance is hyaluronic acid and is present in the material in a final concentration from approximately 1 to approximately 10 mg/ml of material, in particular with 4 mg/ml of material.

Further examples include, but not exclusively, the following synthetic or natural or recombinant sources thereof: growth hormones, including human growth hormone and recombinant growth hormone (rhGH), bovine growth hormones, porcine growth hormones; growth-hormone-releasing hormones; interferons, including interferon-alpha, interferon-beta and interferon-gamma; interleukin-1; interleukin-2; insulin; insulin-like growth factor, including IGF-1; heparin; erythropoietin; somatostatin; somatotropin; protease inhibitors; adrenocorticotropin; prostaglandins; and analogs, fragments, mimetics or polyethylene-glycol (PEG)-modified derivatives of these compounds; or a combination thereof. It is clear that all (active) substances to be released in situ from supports/matrices at the present point in time in the general field of the therapy of diseases are suitable for use in the present invention, it being clear to the skilled worker in each case that the (active) substance to be employed, or the cells to be employed, depend(s) on the respective case to be treated.

In one embodiment of the method according to the invention it is preferred if the serum albumin or the serum protein is functionalized by groups which are selected from among maleimide, vinylsulfonic, acrylate, alkyl halide, azirine, pyridyl, thionitrobenzene acid groups or arylating groups.

In the present context, “functionalized”, or functionalizing, is understood as meaning any—finished—process by which the polymer is imparted a function which it normally does not have—for example by adding groups to the polymer.

By functionalizing the polymer with maleimide groups, it is possible to ensure good crosslinking of the polymer and simultaneously the viability of cells or the biofunctionality of substances when the latter are introduced into the composition/material. The cells or substances which are to be introduced into the composition as the case may be are introduced by dispersing into the composition with the functionalized polymer, which crosslinks with the cells/substances.

As already mentioned further above, the invention also relates to a coating method whereby the composition or the material is/are applied as a coating and/or surface modification of implants which are composed of materials other than the material which is polymerized starting from the abovementioned composition.

Such a coating or modification offers the possibility of coating implants which are composed of a different material which does not have the same degree of compatibility, thereby making these implants, which normally promote endothelial cell proliferation and therefore also angiogenesis, non-angiogenic. In this case, suitable implants are all implants, in particular those which themselves are based on hydrogels, but not on the composition. This is furthermore advantageous in particular in those cases where a direct chemical bonding chemistry as is employed for the polymerized material is possible. This permits the covalent bonding of a thin layer of material to the implant material.

As already mentioned further above, the invention also relates to a method for the treatment or prevention of angiogenesis-associated diseases comprising the step of administering/implanting the above-described composition or of the polymerized hydrogel-material to/in a subject or person in need thereof.

The advantage of this measure is that these diseases can be alleviated or even preventatively prevented by inhibiting angiogenesis by means of the use according to the invention.

A list of angiogenesis-associated diseases can be found, for example, in Carmeliet, “Angiogenesis in health and disease”, Nature Medicine (2003), vol. 9, No. 6: 653-660, and in particular in table 1 specified therein, in which diseases which are characterized by excessive angiogenesis are listed. These include, for example, carcinoma, some infection diseases, autoimmune diseases, DiGeorge syndrome, arteriosclerosis, obesity, psoriasis, Kaposi sarcoma, diabetic retinopathy, primary pulmonary hypertension, bronchial asthma, peritoneal adhesions, endometriosis, arthritis, synovitis, osteophytosis, osteomyelitis.

Further advantages can be seen from the description and the appended drawing.

Naturally, the abovementioned features, and the features yet to be illustrated hereinbelow, may be used not only in the combinations specified in each case, but also in other combinations or alone, without departing from the scope of the present invention.

### BRIEF DESCRIPTION OF THE DRAWINGS

Exemplary embodiments of the invention are shown in the drawing and are explained in greater detail in the description which follows; in which:

FIG. 1 shows the results of adhesion experiments of endothelial cells on a polymerized hydrogel-forming serum-albumin-based material (hereinbelow also referred to as “albumin gel” or “albugel”): schematic representation of the endothelial cell culture on the albugel (A); diagram of the quantitative determination of the number of endothelial cells on the albugel after 1 day and after 5 days (B); phalloidin-stained endothelial cells under the various culture conditions (C);

FIG. 2 shows the detection of the vitality of the endothelial cells on albugel: diagram of the quantitative determination of endothelial cells on the albugel (A); diagram of the investigation of the cytotoxic effect of albugel extracts on endothelial cells (B); calcein- and DAPI-stained endothelial cells under the various culture conditions (C, E, G, I) and uptake of Dil-Ac-LDL (D, F, H, J);

FIG. 3 shows the results of the investigation into the proliferation of endothelial cells on albugel: DAPI- and BrdU-stained endothelial cells under the various culture conditions (A-D); diagram of the quantitative determination of the proliferation of endothelial cells on the albugel (E);

FIG. 4 shows the results of the investigations into the invasion of endothelial cells across the albugel: schematic representation of the structure (A); diagram of the quantitative determination of endothelial cells migrated across the albumin gel (B); diagram of the analysis of the chemotactic index (C); diagram of the analysis of the chemoinvasive index (D); Rose-Bengal-stained endothelial cells on the underside of the transwell filters (E-L);

FIG. 5 shows the results of the investigations into the introgression of blood vessels of the chorioallantoic membrane into the albugel: photographs of the implants in ovo (A, B); photographs of the explanted chorioallantoic membrane with the albugel (C, D); HE—(hematoxylin-eosin) stained chorioallantoic membrane with the albugel (E, F); Sambucus-nigra-lectin-stained chorioallantoic membrane with albugel (G, H); and phase-contrast photographs relating to G and H (I, J);

FIG. 6 shows the results of implantation experiments of albugel subcutaneously into the back of a Scid/nu mouse: HE staining of the albugel with surrounding mouse tissue (A), Sambucus nigra lectin staining and DAPI staining of the albugel with surrounding mouse tissue (B); and

FIG. 7 shows the results of adhesion experiments of immortalized endothelial cells on the albugel: phalloidin-stained endothelial cells under the various culture conditions (A-F); diagram of the quantitative determination of the number of endothelial cells on the albugel after 1 day (G).

### DESCRIPTION OF PREFERRED EMBODIMENTS

### EXAMPLES

A) Preparation of Maleimide-Modified Serum Albumin

250 mg of human, rabbit or ovine serum albumin (Sigma-Aldrich) were dissolved in 5 ml of 1M Na borate (pH 8.2). To this were added 75 μl of a 260 mM N-maleoyl-β-alanin (Sigma-Aldrich cat. no. 63285) solution in PBS/Na borate (pH 8.2) (1:1) and the mixture was incubated for 90 min at room temperature. 106 mg of N-hydroxysuccinimidyl-3-maleimidopropionate (SMP, Obiter Research, Urbana, Ill., USA) were dissolved in 950 μl of dimethylformamide (DMF). Insoluble material was removed by centrifugation. 500 μl of the supernatant were added to the albumin solution, which was thereafter incubated for 60 min at room temperature. Thereafter, 500 μl of 3M sodium acetate (pH 4.7) were added thereto and the mixture was dialyzed three times on ice against 1 liter of PBS. Thereafter, the dialyzate was concentrated by ultrafiltration (YM-3 membrane, Millipore) to a volume of 3.5 ml, filter-sterilized and stored at −80° C.

The serum albumin/protein functionalized thus can be polymerized by addition of SH crosslinkers. A suitable crosslinker in this case is in particular bis-thio polyethylene glycol, which has an SH group at both ends. Besides bis-thio-PEG, other crosslinkers are in general substances which carry SH groups, in particular polymers, and for example dithio-PEG or SH-modified dextran, SH-modified polyvinyl alcohol, SH-modified polyvinylpyrrolidone and the like.

Bis-thio-PEG is commercially available; the crosslinker with a molar mass of 10 000 g/mol was used. If the molar mass is lower, gel formation is reduced, while higher masses result in unduly rapid gelling of the gel, which makes sufficient mixing of the substances impossible. The best gel formation is achieved when SH groups of the crosslinker and maleimide groups of the albumin are present in equimolar concentrations. A final concentration of 3 mM maleimide and SH groups in the gel was used in each case. In addition, the ovine albugel contained 4 mg/ml highly polymeric hyaluronic acid (hereinbelow and in the figures also referred to/abbreviated to “HA”), which is admixed prior to the polymerization reaction and is therefore present in physically firmly anchored form. However, a very wide range of animal and human serum albumins may be used as the albumin source.

B) Experiments on the Detection of the Non-Angiogenic Property of the Albugel

1. Testing the Albugel as a Substrate for Human Endothelial Cells

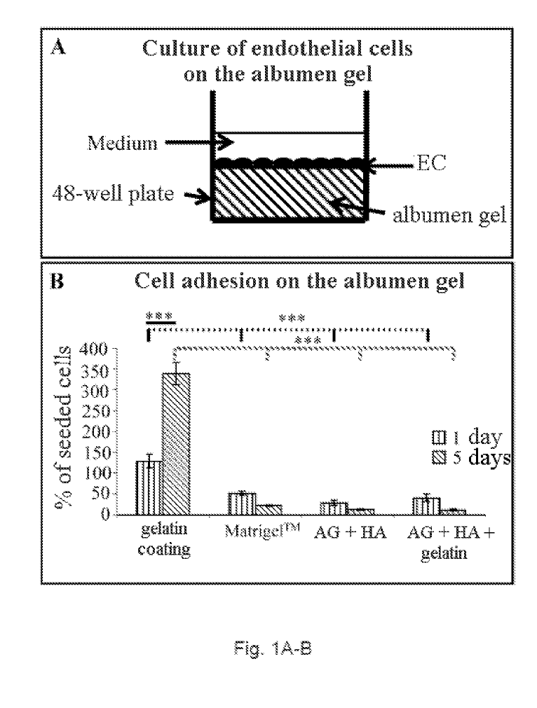

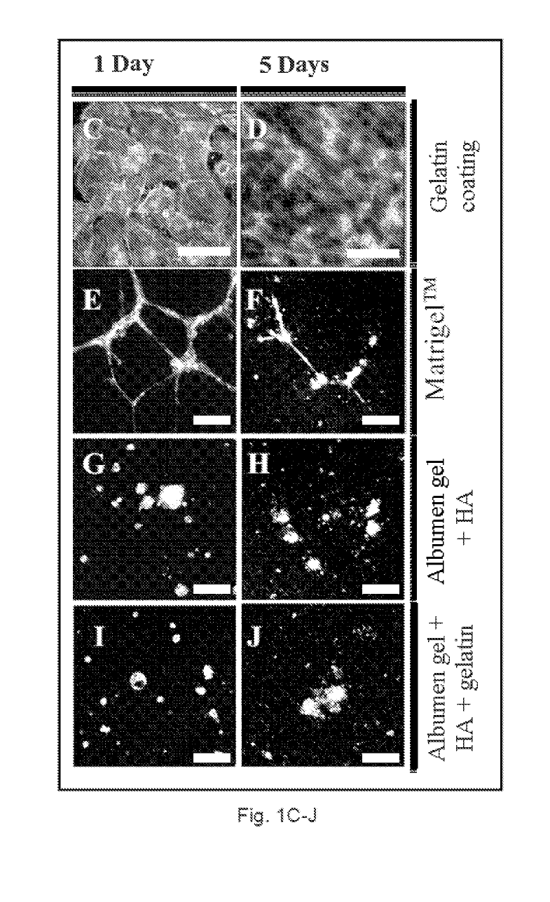

a) To study the effects of albugel on endothelial cells (“EC”), primary human umbilical vein endothelial cells (HUVEC) (PromoCel, Heidelberg) of passages 3 to 9 were cultured on gel, and cell adhesion, cell vitality and cell proliferation were subsequently studied. To this end, in each case 100 μl of ovine albugel were fully polymerized in a 48-well plate, and in each case 1.5×10⁴ HUVECs in 300 μl of endothelial cell medium per well were cultured on the gel for 24 hours or for 5 days (experimental setup, see FIG. 1A). As a control, the same number of cells was cultured in a gelatin-treated (0.5%) 48-well plate and an albugel with an additional 0.5% of gelatin (final concentration in the gel) was prepared. At the same time, the cells were cultured on 10 mg/ml Matrigel™, a little-defined basal membrane extract from Engelbreth-Holm-Swarm murine sarcoma, which acts as a basal membrane equivalent in primary research.

Preparation of the Albugels (Hereinbelow and in the Figures also Abbreviated to/Referred to as “AG”):

| | Ovine serum albumin gel |
|---|---|
| | with hyaluronic acid |
| Mix endothelial cell medium (without FCS) | X × 53 μl |
| with | |
| maleimide-modified ovine serum albumin | X × 7 μl |
| and | |
| Visiol (20 mg/ml) | X × 20 μl |
| introduce bis-thio-PEG into plate and mix | X × 20 μl |
| with gel material | |
| Final volume | X × 100 μl |

### Gelatin Coating and Matrigel™:

For the gelatin coating, 2% of gelatin solution were mixed 1:4 with PBS (phosphate-buffered saline) and the plates were incubated therewith for 30 min. Thereafter, the plates were washed once with PBS. Matrigel™ (20 mg/ml; BD Biosciences, San Jose, USA) was mixed 1:2 with endothelial cell medium without FCS (fetal calf serum) and polymerized fully in the plate for 20 min at 37° C.

The cells were cultured for 1 day or for 5 days under the different cultivation conditions. For the evaluation, the endothelial cells were treated as follows:

After 1 day and after 5 days, the HUVECs were fixed with 2.5% glutaraldehyde/PBS, permeabilized with 0.2% Triton-X 100/PBS and thereafter stained with DAPI (4′-diamidino-2-phenylindole) and Phalloidin Oregon Green, to determine cell adhesion.

Alternative: proliferating HUVECs were visualized after 1 day with the aid of the “5-bromo-2′-deoxyuridine cell labeling and detection kit I” (Roche, Mannheim).

Alternative: after 1 day and after 5 days, the cells were treated with Dil-Ac-LDL (1,1′-dioctadecyl-3,3,3′,3′-tetramethylindocarbocyanine-perchlorate-acetylated low-density lipoprotein) and thereafter stained with calcein and DAPI in order to detect live and dead HUVECs.

b) In Addition, Albugel Extracts were Studied in Respect of their Cytotoxic Effect on Endothelial Cells.

Preparation of the Extracts:

200 μl of albugel (with and without addition of gelatin) were polymerized fully in an Eppendorf vessel and cultured with 1 ml of endothelial cell medium for 24 hours at 37° C., with shaking. As a control, extracts of Matrigel™ were prepared accordingly. The endothelial cells were incubated for 24 hours with the different extracts and with DMSO (dimethyl sulfoxide) as dead control. The vitality of the cells in relation to cells cultured only with medium was determined by means of Alamar Blue assay.

c) Results:

As can be seen from FIG. 1, the cell count after 1 day of culturing on the albugels and on Matrigel™ was markedly lower than the cell count of the gelatin coating. After culturing for 5 days, the cell count on the gels dropped, whereas it continued to rise on gelatin. The cell morphology under the different culture conditions can be seen from FIG. 1C-J. While the endothelial cells formed aggregates on the albugels and were not capable of adhering to the gel, the endothelial cells spread on gelatin and formed typical “tubes” on Matrigel™. The results demonstrate that endothelial cells are not capable of adhering to the albugel and are detached from the gel, when the medium is changed, for example.

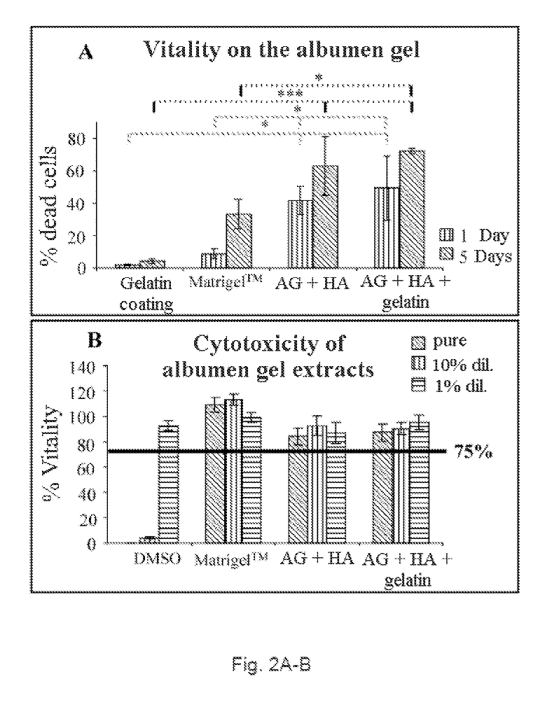

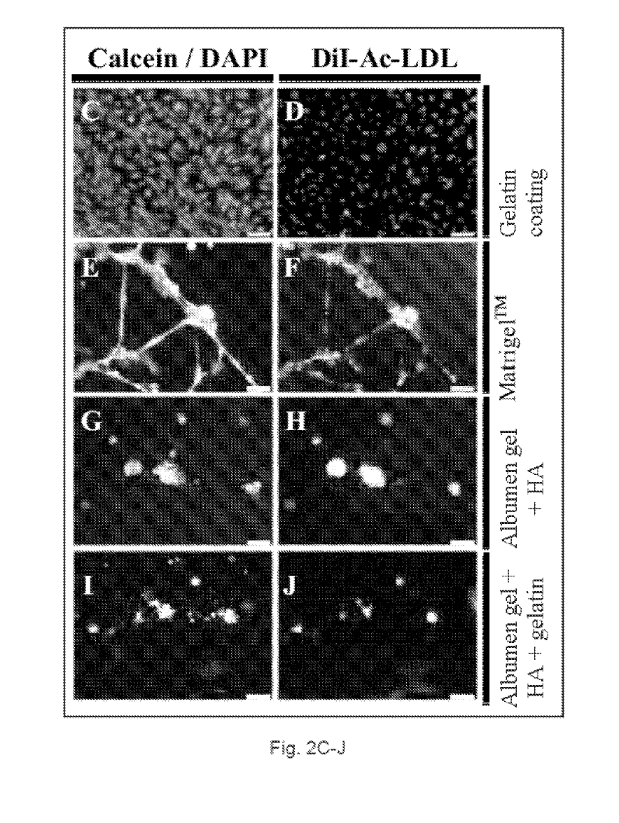

The vitality of the endothelial cells is shown in FIG. 2A and, in qualitative terms, in FIGS. 2C, E, G and I. While hardly any dead cells were detected on the gelatin coating, even after 5 days, and less than 40% of the cells were dead on Matrigel™ after 5 days, the number of dead endothelial cells on the albugels rose to above 60%. In contrast, live cells were capable of taking up Dil-Ac-LDL under all culture conditions (see FIG. 2D, F, H, J), according to which the functionality of the endothelial cells was retained. In addition, albugel extracts had no cytotoxic effect on the endothelial cells (FIG. 2, B).

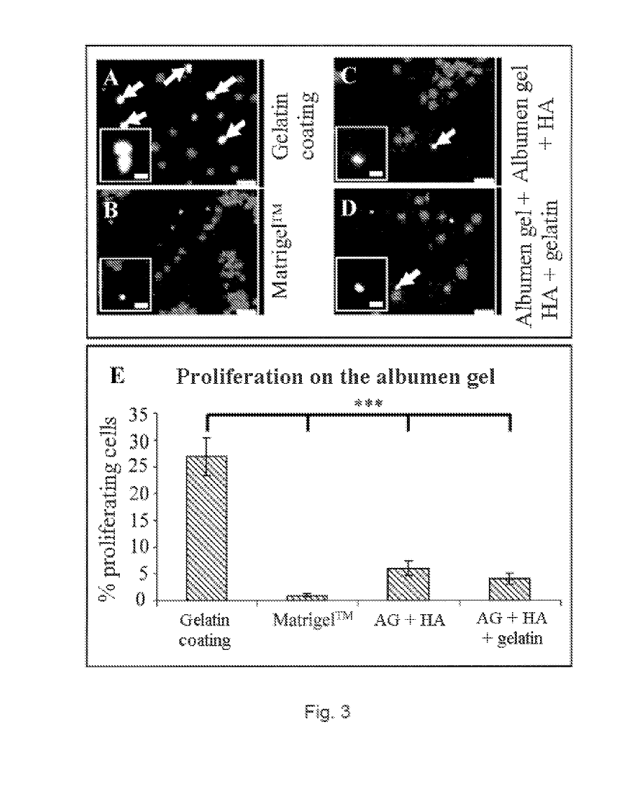

As can be seen from FIG. 3 (A-D qualitative, E quantitative), endothelial cells proliferated only to a minor extent on the albugel in contrast to the gelatin coating.

The addition of gelatin, which is known for its proangiogenic and proadhesive properties, to the albumin gel had no positive effect on the endothelial cells.

2. Testing the Albugel as a Substrate for Human Endothelial Cells

To rule out that hyaluronic acid (“HA”) is responsible for the observed effects, the following experiments were carried out with rabbit albugel without hyaluronic acid.

a) Procedure:

Immortalized human umbilical vein endothelial cells (HUVEC hTERT) were used for the culture.

1.5×10⁴ HUVECs were cultured in 300 μl of endothelial cell medium on 0.5% gelatin coating, 100 μl of Matrigel™ (10 mg/ml) or 100 μl of pure albumin gel in a 48-well plate. A further control was prepared additionally with 0.5% of gelatin (final concentration).

Preparation of the Albumin Gels:

| | Rabbit serum albumin gel |
|---|---|
| Mix endothelial cell medium | X × 73 μl |
| (without FCS) with | |
| maleimide-modified ovine serum | X × 7 μl |
| albumin and | |
| introduce bis-thio-PEG into plate | X × 20 μl |
| and mix with gel material | |
| Final volume | X × 100 μl |

Gelatin Coating and Matrigel™:

For the gelatin coating, 2% of gelatin solution were mixed 1:4 with PBS (phosphate-buffered saline) and the plates were incubated therewith for 30 min. Thereafter the plates were washed once with PBS.

Matrigel™ (20 mg/ml) was mixed 1:2 with endothelial cell medium without FCS (fetal calf serum) and polymerized fully in the plate for 30 min at 37° C. The cells were cultured for 1 day under the different culture conditions.

For the evaluation, the endothelial cells were treated as follows:

after 1 day the HUVECs were fixed with 2.5% glutaraldehyde/PBS, permeabilized with 0.2% Triton-X 100/PBS and thereafter stained with DAPI (4′,6-diamidino-2-phenylindole) and Phalloidin Oregon Green, to determine cell adhesion.

b) Results:

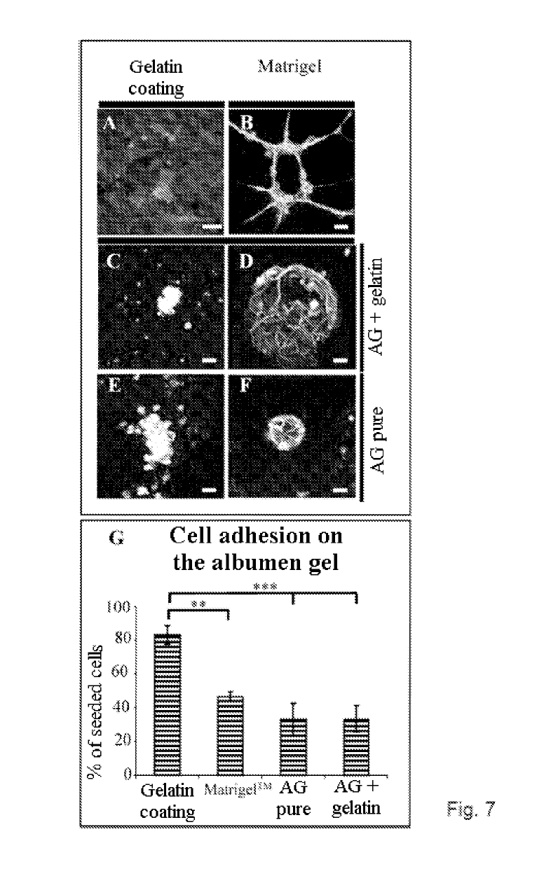

As can be seen from FIG. 7G, the cell count after 1 day of culturing on the albugels and on Matrigel™ was considerably lower than the cell count of the gelatin coating. The cell morphology under the different culture conditions can be seen from FIG. 7A-H. The endothelial cells spread on gelatin and form typical “tubes” on Matrigel™ Endothelial cells on the albugel develop aggregates (FIGS. 7C and E) or a spheroid-like structure (FIGS. 7D and F).

3. Invasion of Endothelial Cells Across the Albugel

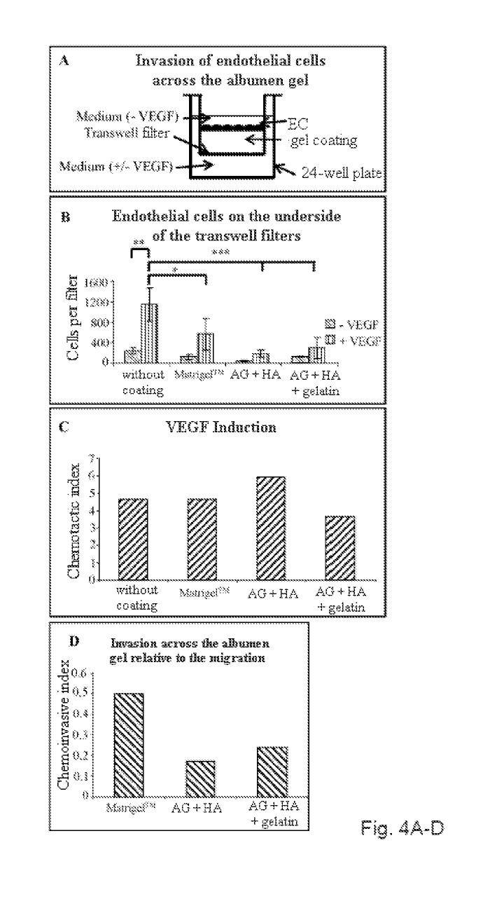

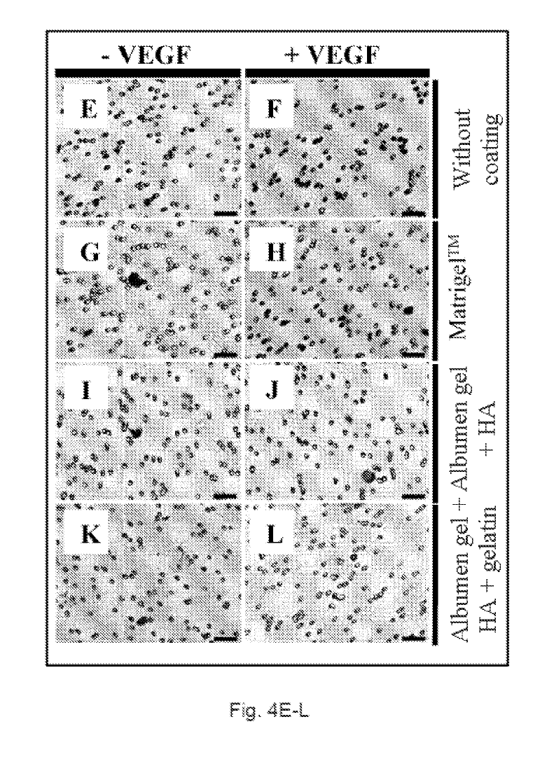

a) The invasion of endothelial cells across the albugel on a transwell filter was compared with the invasion across Matrigel™ and the migration across an uncoated filter. A schematic representation of the experimental setup can be seen from FIG. 4A.

Transwell filters with a pore size of 8 μm were coated with either 100 μl of albugel with hyaluronic acid, 100 μl of albugel with hyaluronic acid and with 0.5% gelatin, or with 100 μl of Matrigel™ (5 mg/ml), respectively. Uncoated transwell filters were used for determining the migration.

For each coating, 3×10⁵ Hoechst 33258-labeled HUVECs in 200 μl of endothelial cell medium were transferred to the filters. After the cells had settled for 2 hours, 600 μl of endothelial cell medium with and without 40 ng/ml VEGF (vascular endothelial growth factor) were pipetted into the bottom compartment. After 24 hours, the cells on the upper side of the filter were wiped off and the cells on the underside of the filter were fixed and counted. As an alternative, the cells were stained with Rose Bengal.

b) Results:

The number of endothelial cells on the underside of the transwell filters is shown in FIG. 4B, Rose-Bengal-stained endothelial cells in FIG. 4E-L. The chemotactic index (see FIG. 4C), which specifies the quotient of migration or invasion with VEGF induction to without VEGF induction, and the chemoinvasive index (see FIG. 4D), which specifies the quotient of cells migrated and moved across a gel, were calculated from the means of the cell counts on the underside of the filters. The chemotactic indices of all coatings were approximately equally high; accordingly, the induction by VEGF is comparable. However, the chemoinvasive indices of the two albugels were markedly below the chemoinvasive index of Matrigel™, which demonstrates that endothelial cells are only capable of a minor degree of migration across the albugel.

4. Introgression of Blood Vessels of the Chorioallantoic Membrane into the Albugel

a) Procedure

Eggs of the breed Hissex Braun were provided on embryonal day 3 with a window in the eggshell. On embryonal day 8, 200 μl of albugel with hyaluronic acid and 200 μl of albugel with hyaluronic acid and 0.5% of gelatin were transferred to the chorioallantoic membrane (CAM), which showed a high degree of vascularization. Incubation of the eggs was continued to embryonal day 13. The CAM was fixed in ovo in 4% PFA (paraformaldehyde)/PBS at room temperature, then explanted and fixed for a further day at 4° C., dehydrated for 2 days in 30% sucrose/distilled water and frozen in Tissue Tek (O.C.T. Compound, Sakura; Torrance, Canada). Frozen sections with a thickness of 7 μm were stained with HE (hematoxylin-eosin) and frozen sections with a thickness of 5 μm with the Sambucus nigra lectin.

b) Results:

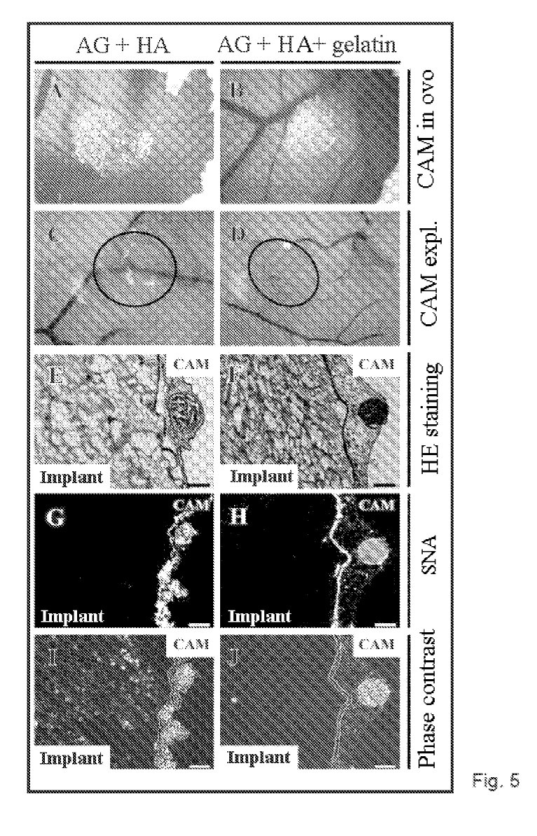

The blood vessels of the CAM do not grow toward the implanted albugels (FIG. 5A-D). Blood vessels were detected neither in HE-stained (FIGS. 5E and F) nor in Sambucus-nigra-lectin-stained sections (FIGS. 5G and H), whereas blood vessels were detected in the CAM with the aid of both staining methods. The results demonstrate that the albugel had no angiogenic influence on the blood vessels of the CAM.

5. Implantation of the Albugel into the Back of a Scid/nu Mouse

a) Albumin gels based on human serum albumin were populated with human intervertebral-disk cells and injected into the backs of Scid/nu mice. Two weeks after the implantation, the albumin gels were reexplanted, and sections of these explanted albumin gels were HE-stained and then examined. In addition, blood vessels were detected by means of an immunohistochemical staining against human von Willebrand factor.

b) Results

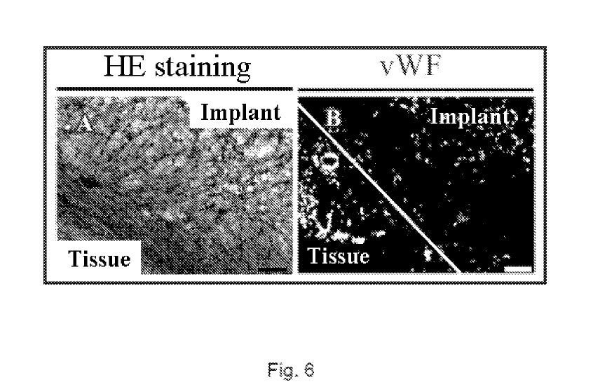

No blood vessels were detected by means of HE staining, neither in the surrounding murine tissue nor in the implants (FIG. 6A), while the nuclei of human intervertebral-disk cells in the implant were stained by hematoxylin. The specific staining of blood vessels with an antibody against human von Willebrand factor demonstrated that, while blood vessels were located in the surrounding tissue, the vessels did not introgress into the albugel (FIG. 6B). Only the DAPI-stained human intervertebral-disk cells are discernible in the implant.

In summary, it was possible to demonstrate with the above-described experiments that endothelial cells scarcely adhere to or proliferate on albugel. In addition, it was demonstrated that endothelial cells die on the albugel, but not due for instance to any toxicity of the albugel, but, rather, due to the lack of cell adhesion, which is imperative for survival. Also, addition of the chemotactic attractant VEGF failed to provoke migration of the endothelial cells into the albugel, nor did blood vessels of the chicken egg chorioallantoic membrane migrate into the albugel; nor did in vivo experiments on mice with the albugel show any migration of blood vessels into the albugel.

Thus, these non-permissive properties in respect of endothelial cells open up the potential of using the albugel as a matrix/implant for inhibiting and preventing angiogenesis and the adhesion of endothelial cells, in particular in the implantation field of medicine, for example in the treatment of scleroses, and in regenerative medicine, for example in the treatment of diseased and/or defective cartilage tissue, intervertebral-disk tissue and cornea tissue.

What is claimed is:

1. A method for inhibiting and/or preventing angiogenesis or endothelial cell proliferation, comprising the step of administering to a subject in need thereof a biocompatible composition, which composition is polymerizable to a hydrogel-material and which is based on a hydrophilic polymer, wherein the hydrophilic polymer is crosslinkable serum albumin or crosslinkable serum protein.

2. The method as claimed in claim 1, wherein the composition includes at least one of the following: mammalian cells, a pharmacological agent, a biologically active agent, or one or more or mixtures of these.

3. The method as claimed in claim 1, wherein the serum albumin or the serum protein is functionalized by groups which are selected from among maleimide, vinylsulfonic, acrylate, alkyl halide, azirine, pyridyl, thionitrobenzene acid groups or arylating groups.

4. The method as claimed in claim 1, wherein the serum albumin and the serum protein are human serum albumin and human serum protein.

5. The method as claimed in claim 2, wherein the pharmacologically active agent is selected from at least one of the following: an antibiotic, an anti-inflammatory, a metabolism hormone, chondroprotectants, agents for gene therapy, growth hormones, differentiation or modulation factors, immunosuppressants, immunostimulants, DMOAs, nucleic acids, apoptosis-inducing active substances, adhesion-mediating active substances, receptor agonists and receptor antagonists, or mixtures of these.

6. The method as claimed in claim 1, wherein the albumin concentration in the material is from approximately 5 to approximately 15 mg/ml material, in particular approximately 10 mg/ml material.

7. The method as claimed in claim 1, wherein the composition is administered in injectable form.

8. The method as claimed in claim 1, wherein the hydrogel-material is administered as surface coating of an implant.

9. The method of claim 1, wherein the angiogenesis is inhibited and/or prevented in in the treatment of scleroses, of diseased and/or defective cartilage tissue, diseased and/or defective intervertebral-disk tissue, diseased and/or defective cornea tissue, of carcinoma, infection diseases, autoimmune diseases, DiGeorge syndrome, arteriosclerosis, obesity, psoriasis, Kaposi sarcoma, diabetic retinopathy, primary pulmonary hypertension, bronchial asthma, peritoneal adhesions, endometriosis, arthritis, synovitis, osteophytosis, osteomyelitis.

10. A method for inhibiting and/or preventing angiogenesis or endothelial cell proliferation in a subject in need thereof, comprising the step of administering a polymerized hydrogel-material, which has been obtained by polymerizing a serum-albumin- or serum-protein-based composition.

11. The method as claimed in claim 10, wherein the material is administered in the form of an implant.

12. The method as claimed in claim 10, wherein the polymerized material is administered as the surface coating of an implant.

13. The method as claimed in claim 10, wherein the serum albumin and the serum protein are human serum albumin and human serum protein.

14. The method of claim 10, wherein the angiogenesis is inhibited and/or prevented in in the treatment of scleroses, of diseased and/or defective cartilage tissue, diseased and/or defective intervertebral-disk tissue, diseased and/or defective cornea tissue, of carcinoma, infection diseases, autoimmune diseases, DiGeorge syndrome, arteriosclerosis, obesity, psoriasis, Kaposi sarcoma, diabetic retinopathy, primary pulmonary hypertension, bronchial asthma, peritoneal adhesions, endometriosis, arthritis, synovitis, osteophytosis, osteomyelitis.
